(19) United States
(12) Patent Application Publication
(10) Pub. No.: US 2001/0005720 A1
(43) Pub. Date: Jun. 28, 2001

(54) METHOD OF TREATING CHRONIC PROGRESSIVE VASCULAR SCARRING DISEASES

(75) Inventors: GARY E. STRIKER, MIAMI, FL (US); LILIANE J. STRIKER, MIAMI, FL (US)

Correspondence Address: MARTIN W SCHIFFMILLER, KIRSCHSTEIN OTTINGER ISRAEL &, SCHIFFMILLER, 489 FIFTH AVENUE, NEW YORK, NY, 100176105

(73) Assignee: U.S.A. AS REPRESENTED BY THE SECRETARY DEPARTMENT OF HEALTH AND HUMAN SERVICES

(21) Appl. No.: 08/840,777

(22) Filed: Apr. 16, 1997

Related U.S. Application Data
(63) Continuation-in-part of application No. 08/478,347, filed on Jun. 7, 1995, now Pat. No. 5,643,892.

(51) Int. Cl. A61K 31/715; A01N 43/04

(52) U.S. Cl. 514/54

(57) ABSTRACT

A method of treating a mammalian patient suffering from a chronic progressive vascular scarring disease (CPVSD), particularly arteriosclerotic diseases such as atherosclerosis, to halt or at least slow substantially the progress of the disease and cause resolution and/or diminution of already-formed scarring and lesions. The method consists of the administration to the patient of a pharmaceutical composition containing an effective amount of pentosan polysulfate (PPS) or a pharmaceutically acceptable salt thereof. The oral route of administration is preferred, with the total daily dosage of PPS or PPS salt ranging from about 5 to about 30 mg/kg of patient body weight, or about 350 to about 2,000 mg per day in adult human patients.

### CROSS-REFERENCE TO RELATED APPLICATION

This application is a continuation-in-part of co-pending application Ser. No. 08/478,347, filed Jun. 7, 1995.

### BACKGROUND OF THE INVENTION

1. Field of the Invention

The invention relates to methods and pharmaceutical compositions used to treat chronic progressive vascular scarring diseases.

2. Description of the Prior Art

Chronic progressive vascular scarring disease (CPVSD) is a complication of several of the most common diseases afflicting the developed world, including diabetes mellitus, hypertension, the various hyperlipidemias, and the like. The present therapeutic modalities dealing with CPVSD are aimed at the underlying causes. Unfortunately, for the most part there are no known cures, or their control is very difficult to accomplish in the general population. In addition, CPVSD is often not only well-established, but also far-advanced, by the time that the underlying cause(s) come to medical attention. Thus, one is left with attempting to treat secondary complications, of which CPVSD is the most serious because it leads to renal failure, strokes, heart disease and blindness.

Generally, CPVSD is characterized by a change in vascular smooth muscle cells. One of the major changes is an increase in the amount and alteration of the types of connective tissue that they synthesize. This results in scarring and marked changes in function. In blood vessels, this leads to loss of elasticity, resulting in vessels which do not distend and contract and which have thickened walls and narrowed lumens. The end result is reduced blood flow or complete blockage. Examples of vascular scarring diseases characterized by these pathophysiological processes include chronic progressive glomerular disease, e.g., diabetic-induced glomerulosclerosis (scarring); progressive renal failure after renal transplantation; occlusion of shunts used to provide vascular access in patents with end stage renal disease being treated with hemodialysis; other chronic small blood vessel diseases (such as in some patients with hypertension); recurrence of stenosis in patients who have undergone coronary bypass surgery; and diabetic retinopathy.

The therapeutic goal of any treatment for CPVSD must be to decrease the already-formed excess of extracellular matrix (scarring) in order to restore normal vessel patency and function, or at the very least prevent or substantially slow further progression. However, there is currently no direct method of interfering with abnormalities in smooth muscle tissue metabolism or to modulate connective tissue synthesis, despite their importance in chronic progressive disease. Progression of these diseases has been considered to be both inevitable and irreversible.

It is, therefore, particularly important that a treatment regimen be developed for CPVSD, preferably involving oral administration of a pharmaceutical agent of low toxicity, which is effacious in treating and reversing CPVSD by causing regression and degradation of established lesions.

Pentosan polysulfate (PPS) is a highly sulfated, semisynthetic polysaccharide with a molecular weight ranging from about 1,500 to 6,000 Daltons, depending on the mode of isolation. PPS may be in the same general class as heparins and heparinoids, but there are a number of differences in chemical structure, methods of derivation and physico-chemical properties between.-PPS and heparin. While heparin is usually isolated from mammalian tissues such as beef and pork muscles, liver and intestines, PPS is a semi-synthetic compound whose polysaccharide backbone, xylan, is extracted from the bark of the beech tree or other plant sources and then treated with sulfating agents such as chlorosulfonic acid or sulfuryl trichloride and acid. After sulfation, PPS is usually treated with sodium hydroxide to yield the sodium salt.

As illustrated by the following formulas,

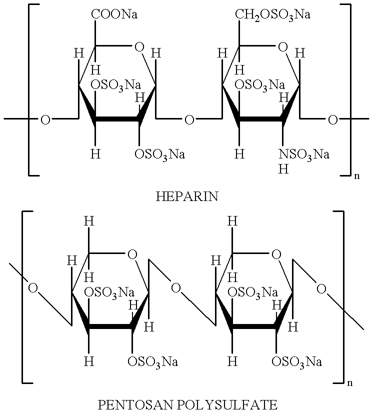

heparin is a sulfated polymer of repeating double sugar monomers, (D)-glucosamine and (D)-glucuronic acid (both 6-carbon hexose sugars), with an amine function on the glucosamine; PPS is a sulfated linear polymer of repeating single monomers of (D)-xylose, a 5-carbon pentose sugar in its pyranose ring form. While heparin rotates plane polarized light in a dextrorotatory direction, PPS rotates light in a levorotatory direction.

In terms of biological properties, PPS prolongs partial thromboplastin time and has been used to prevent deep venous thrombosis, but it has only about one-fifteenth the anticoagulant potency of heparin (see generally Wardle, J. Int. Med. Res., 20:361-370, 1992). PPS has also been disclosed as useful in the treatment of urinary tract infections and interstitial cystitis (U.S. Pat. No. 5,180,715) and, in combination with an angiostatic steroid, in arresting angiogenesis and capillary, cell or membrane leakage (U.S. Pat. No. 4,820,693).

Some researchers have demonstrated that PPS inhibits smooth muscle cell proliferation and decreases hyperlipidemia, and on that basis have suggested that PPS might be useful prophylactically in limiting atherosclerotic plaque formation, inhibiting mesangial cell proliferation and preventing collagen formation and glomerulosclerosis (Paul et al., Thromb, Res., 46:793-801, 1987; Wardle, ibid.). However, no one had previously focused on the scarring aspects of CPSVD (as opposed to inhibition of cell proliferation) such as atherosclerosis or demonstrated that it was feasible to halt and/or reverse vascular scarring, i.e., PPS had not been considered in this context. Moreover, none of the prior art suggestions of the possible utility of PPS in scarring diseases was supported by any substantial scientific efficacy data generated in intact animals, but instead were based on in vitro studies of animal tissue which are frequently not predictive of in vivo efficacy.

Although there have recently been disclosures of the utility of PPS in the inhibition of fibrosis and scar formation (see, e.g., Roufa et al., U.S. Pat. No. 5,605,938), these teachings deal with the suppression of fibroblast invasion in skin and related tissue areas, but not scarring diseases of smooth muscle cells which are very different in etiology and pathology.

### SUMMARY OF THE INVENTION

It is the object of the present invention to provide a method of treating CPVSD not only to halt the disease process but to actually reverse that process and cause the regression of existing scarring or lesions. It is a further object of the invention to provide such a method of treatment utilizing a commercially available pharmaceutical agent which may be administered by conventional means, which is non-toxic and not likely to provoke serious side effects and which is highly efficacious in treating CPVSD.

In keeping with these objects and others which will become apparent hereinafter, the invention resides, briefly stated, in a method of treating a mammalian patient suffering from CPVSD, to halt the progress of the disease and to cause the resolution or diminution of already-formed scarring or fibrotic lesions in the affected organ or vasculature said method consisting of the administration to the patient of a pharmaceutical composition containing an effective vascular scarring disease treatment amount of pentosan polysulfate or a pharmaceutically acceptable salt thereof. Oral administration of PPS, e.g., in the form of tablets, capsules or liquids, is the preferred mode of administration.

### BRIEF DESCRIPTION OF THE DRAWINGS

FIG. 1 reflects the quantitation of α₁IV collagen mRNA by competitive PCR on one-tenth of a glomerulus from a normal five-week old mouse (as described in Example 1, below), depicting:

a) in its top panel, the reaction scheme and a corresponding ethidium bromide stained gel after PCR amplification; and

b) in its lower panel, a graph plotting the ratio of mutant collagen CDNA per glomerulus against the amount of mutant cDNA inputted into each of nine tubes containing all of the PCR reagents.

FIG. 2 depicts:

a) in its upper panel, PAS-stained kidney sections from two nephrectomy specimens with renal carcinoma (A-normal glomerular histology; B-marked sclerosis);

b) in its middle panel (C-D), immunofluorescence microscopy, antibody to type IV collagen in the same kidneys; and

c) in its lower panel (E), a bar graph reflecting the sclerosis index in the same kidneys; α₂IV collagen CDNA was determined by competitive PCR quantitation of in pools of 50 microdissected glomeruli (values are: 145±22 vs. 1046±74×10⁻⁴ attomoles/glomerulus).

FIG. 3 is a bar graph reflecting the sclerosis index in the kidneys of five human patients without glomerular sclerosis compared to five patients with sclerosis, expressed in glomerular relative cell numbers and α₂IV collagen cDNA levels.

FIG. 4 is a bar graph reflecting α₂/α₃IV collagen mRNA ratios from human patients with membranous glomerulonephritis (MN) and diabetic nephropathy (DM) and from nephrectomies with glomerulosclerosis (NX GS) and without glomerulosclerosis (NX N1).

FIG. 5 is a bar graph reflecting the effect of PPS sodium on DNA synthesis in normal mesangial cells as determined by tritiated thymidine incorporation (24 hours of incubation) and plotted as tritiated counts per minute per 10³ cells vs. concentration of PPS sodium in μg/ml.

FIG. 6 is a bar graph reflecting the effect of PPS sodium on cell growth in normal mesangial cells, plotting cell number after three days of incubation vs. added concentration of PPS sodium in μg/ml.

FIG. 7 is a bar graph reflecting a comparison of the effects of PPS sodium and heparin (with an untreated control group) on cell growth in normal mesangial cells after three and five days of incubation.

FIG. 8 is a graph reflecting normal mesangial cell proliferation over time in cells incubated with serum and PPS sodium compared to control cells incubated only with serum.

FIG. 9 is a chart of MRNA values from normal mesangial cell layers exposed to PPS sodium (100 μg/ml) for varying periods and reverse-transcribed, reflecting the increase, decrease or lack of change in levels of α₁IV and α₁I collagen mRNA, collagenases (metalloproteinases) 72 KDa and 92 KDa mRNA, growth factor TGF-β mRNA and cell protein β-actin mRNA.

FIG. 10 is a bar graph reflecting the ratio of α₁IV collagen/GAPDH, as determined by competitive PCR, elaborated respectively by glomeruli from GH transgenic mice administered PPS sodium in drinking water for 10-12 weeks and glomeruli from control GH mice receiving untreated water.

FIG. 11 depicts photographs of cross-sections of the abdominal aortae of a euthanized Watanabe rabbit from the an untreated control group and another Watanabe rabbit from a group treated with subcutaneous PPS sodium (Elmiron®).

FIG. 12 is a bar graph reflecting the cross-sectional areas of the intima of various branches of the aortae of Watanabe rabbits receiving a high cholesterol diet alone and the intimal areas of comparable cross-sections taken from another group of Watanabe rabbits receiving a high cholesterol diet and PPS sodium in their drinking water

FIG. 13 is a bar graph reflecting the ratios of the intimal to medial cross-sectional areas of various branches of the aortae of Watanabe rabbits receiving a high cholesterol diet alone and the comparable ratios measured in cross-sections taken from another group of Watanabe rabbits receiving a high cholesterol diet and PPS sodium in their drinking water.

### DETAILED DESCRIPTION OF THE INVENTION

The present invention relates to a method of treating a mammalian patient suffering from a chronic progressive vascular scarring disease (CPVSD) in an affected vasculature, particularly an artery such as the aorta, to halt or substantially slow the progress of the disease and cause the resolution and/or diminution of already-formed scarring lesions. The subject method consists of the administration to the patient of a pharmaceutical composition containing an effective vascular scarring disease treatment-amount of pentosan polysulfate (PPS) or a pharmaceutically acceptable salt thereof.

The diseases which may be treated in accordance with the novel method include, but are not limited to, chronic progressive glomerular disease, including scarring-type diabetic-induced glomerulosclerosis; arterial scarring due to arteriosclerosis, including atherosclerosis; progressive renal failure due to interstitial scarring following renal transplantation; occlusion by scarring of shunts used to provide vascular access in patents with end stage renal disease being treated with hemodialysis; other chronic scarring small blood vessel diseases (such as in some patients with hypertension); recurrence of stenosis due to scarring in patients who have undergone coronary bypass surgery; and diabetic retinopathy.

Of particular importance, because of the prevalence and pernicious nature of the disease, is the treatment by the novel method of chronic arteriosclerotic scarring pathologies to reverse or prevent the disease process and resolve existing vascular scarring and lesions. For example, the administration of PPS in accordance with the invention can halt and reverse the progress of atherosclerosis in major vessels, causing the resolution and/or diminution of already-formed scarring involving arterial walls affected by atherosclerotic plaques and substantially increasing the intimal cross-sectional area to allow greater blood flow through the vascular lumen.

The phrase “an effective vascular scarring disease treatment amount” as used herein refers to an amount of PPS or salt thereof incorporated into a pharmaceutical composition which is effective when given one or more times daily for a prescribed period of time in halting and reversing the progressive symptoms of CPVSD. In human patients, a total daily dosage of about 5 to about 30 mg/kg of patient body weight, or about 350 to about 2,000 mg per day in adult patients and preferably about 500 to about 1,500 mg of PPS or PPS salt, said daily dosage being administered in one to four equally divided doses, is effective in achieving the therapeutic goal of treating and reversing CPVSD. In smaller mammals, the dosage range may have to be adjusted downward in accordance with body weight, species and the nature of the condition.

The preferred embodiment of the novel method of treatment is the administration to the patient of a pharmaceutical composition comprising an effective amount of PPS and at least one pharmaceutically acceptable inert ingredient. The composition may be in any standard pharmaceutical dosage form, but is preferably an orally administered dosage form.

Dosage forms for oral delivery may include conventional tablets, coated tablets, capsules or caplets, sustained release tablets, capsules or caplets, lozenges, liquids, elixirs or any other oral dosage form known in the pharmaceutical arts.

As pharmaceutically acceptable inert ingredients there are contemplated fillers, binders, solvents, etc. which do not interfere with the CPVSD treatment activity of the PPS. Also, fillers such as clays or siliceous earth may be utilized if desired to adjust the size of the dosage form.

Further ingredients such as excipients and carriers may be necessary to impart the desired physical properties of the dosage form. Such physical properties are, for example, release rate, texture and size. Examples of excipients and carriers useful in oral dosage forms are waxes such as beeswax, castor wax, glycowax and camauba wax, cellulose compounds such as methylcellulose, ethylcellulose, carboxymethylcellulose, cellulose-acetate phthalate, hydroxypropylcellulose and hydroxypropylmethylcellulose, polyvinyl chloride, polyvinyl pyrrolidone, stearyl alcohol, glycerin monstearate, methacrylate compounds such as polymethacrylate, methyl methacrylate and ethylene glycol dimethacrylate, polyethylene glycol and hydrophilic gums.

In the compositions of the present invention the PPS active ingredient is desirably present in an amount between about 50 and about 300 mg per dosage unit. The exact dosage administered to each patient will be a function of the condition being treated and the physical characteristics of the patient, such as age and body weight.

The active pharmaceutical ingredient can be PPS or a pharmaceutically acceptable salt thereof, e.g., the sodium salt. One preferred oral dosage form for use in the method of the invention is Elmiron® gelatin capsules (Baker Norton Pharmaceuticals, Inc., Miami, Fla.) which contain 100 mg of PPS sodium and, as excipients, microcrystalline cellulose and magnesium stearate.

Although the oral route of administration is preferred, the present method of treatment also comprehends the administration of PPS or a salt thereof via the parenteral, transdermal, transmucosal routes or via any other routes of administration known and conventionally utilized in the medical and pharmaceutical arts. Likewise, the compositions of the invention may include PPS in pharmaceutically acceptable parenteral, transdermal, transmucosal or other conventional vehicles and dosage forms together with suitable inert solvents, excipients and additives. Many examples of such pharmaceutically acceptable vehicles can be found in Remington's Pharmaceutical Sciences (17th edition (1985)) and other standard texts. Whatever route of administration or type of pharmaceutical dosage form is used, the dosage range for the PPS active ingredient is from about 5 to about 30 mg/kg of patient body weight or about 350 to about 2,000 mg, and preferably about 500 to about 1,500 mg, although dosage amounts towards the lower end of that range would probably be utilized on parenteral administration.

The pharmaceutical compositions used in the method pf the invention may include active ingredients other than PPS or a PPS salt, for example, other agents which may be useful in the management of CPVSD.

The novel method enables convenient, safe and effective treatment of patients suffering from various forms of CPVSD which in many instances may be life or organ threatening, By the subject method a pharmaceutical agent proven to have low toxicity and a low incidence of side effects can be used to not only halt what has long been considered the inexorable progress of chronic vascular scarring disease, but actually arrest and/or reverse already-formed scarring lesions to restore normal vessel patency and function.

The following examples include (a) descriptions of experiments already published in the medical literature which validate the use of certain competitive PCR (polymerase chain reaction) techniques for the quantitation of scarring-type collagen mRNA and related factors in glomeruli, and which demonstrate that relative glomerular cell numbers do not correlate with levels of production of scarring-type collagen; (b) experiments conducted by or under the supervision of the inventor which demonstrate in vitro and in vivo the efficacy of PPS in down-regulating the production of scarring-type collagen and cell growth factors and up-regulating collagenese activity to degrade existing deposits of scarring collagen; and (c) experiments conducted by or under the supervision of the inventor which demonstrate in vivo the efficacy of PPS in reversing atherosclerosis, including reducing substantially the amount and distribution of atherosclerotic plaques in afflicted vessels. These examples are not intended, however, to set forth materials, techniques or dosage ranges which must be utilized in order to practice the present invention, or to limit the invention in any way.

### EXAMPLE 1

### QUANTITATION OF COLLAGEN

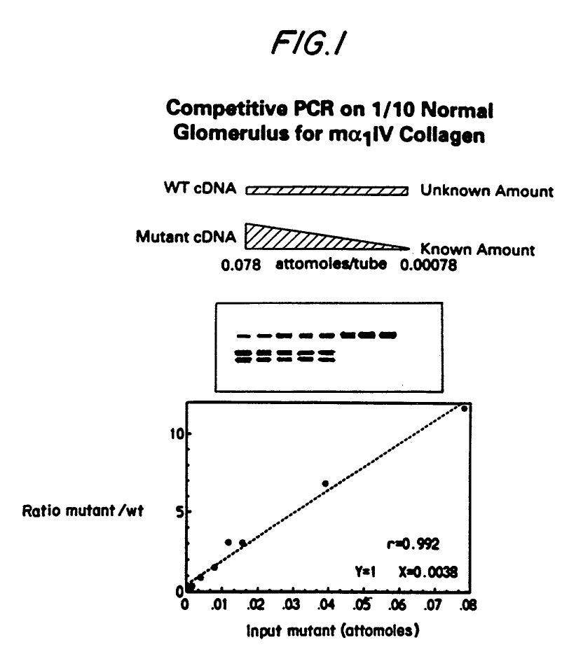

As described in Peten et al. Am. J. Physiol. 32: F951-957 (1992), α₁IV and α₂IV collagen in mouse glomeruli can be quantitated by the following method: the amount of cDNA representing the mRNA in one-tenth of a glomerulus from a normal five-week old mouse and a standard amount of α₁IV or α₂IV collagen primers were added to each of several tubes containing all PCR reagents from the GeneAmp DNA Amplification Kit (PerkinElmer Cetus, Norwalk, Conn.). Serial dilutions of mutated cDNA containing either a new restriction enzyme cleavage site or a deletion were added to this mixture prior to amplification (scheme shown in FIG. 1, top panel). The concentrations of the mutant were determined in a prior experiment designed to bracket the equivalence point (y=1).

After PCR amplification, the entire reaction mix was loaded directly onto a 4% Nusieve: Seakem (3:1) (FMC Bioproducts, Rockland, Me.) agarose gel in a H5 Horizon gel apparatus (Life Technologies) and subjected to electrophoresis. DNA bands were visualized with ethidium bromide staining and ultraviolet (UV) transillumination. Photographs were taken with positive/negative 55 Polaroid films (Polaroid, Cambridge, Mass.) (see FIG. 1, middle panel). Gel negatives were scanned by one-dimensional laser densitometry, for competitive PCR analyses (Shimadzu; Scientific Instruments, Columbia, Md.).

The densitometric values of the test and the mutant band(s) were calculated, and their ratio for each reaction tube was plotted as a function of the amount of mutant template added (FIG. 1, bottom panel). For the α₂IV collagen mutant, the measured densitometric band intensity was corrected by a factor of 562/479 before plotting the mutant/test band ratio. For α₂IV the mutant bands, their densitometric values were added before division by the wild-type (test) band value. A straight line was drawn by linear regression analysis. The quantity of CDNA in the test sample was calculated to be that amount at which the mutant/test band density ratio was equal to 1. Competitive PCR assays were performed in duplicate or triplicate.

### EXAMPLE 2

### CHANGES IN SCLEROTIC GLOMERULI

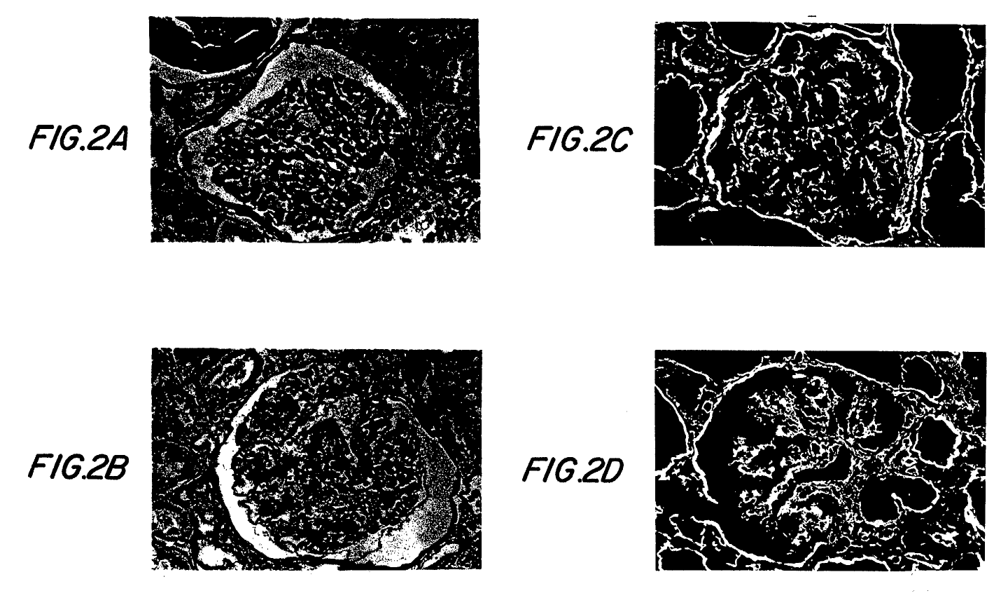

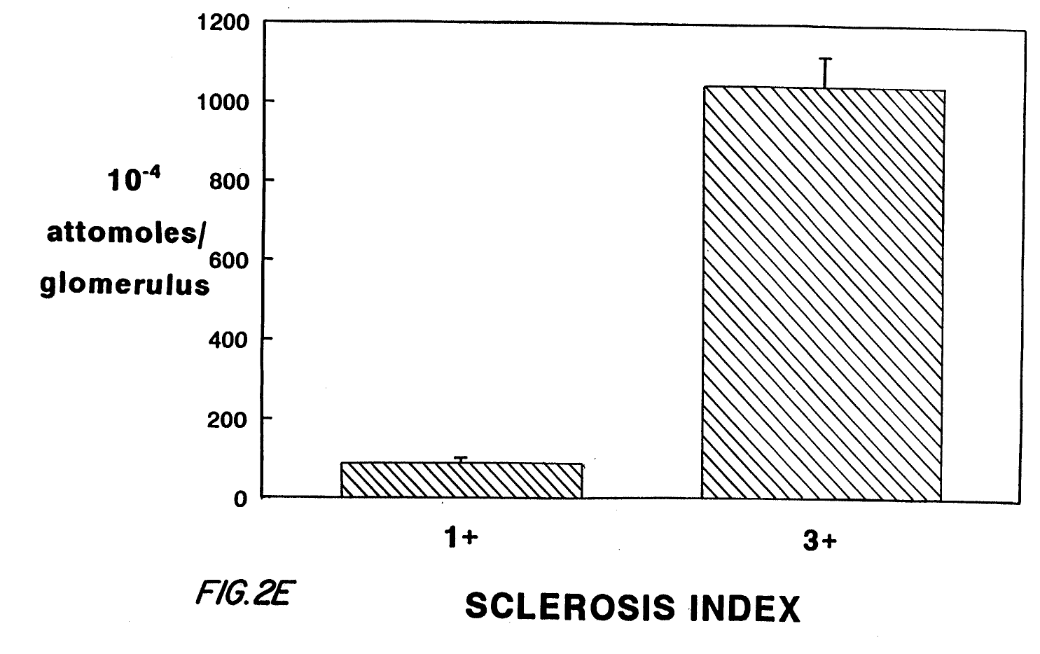

As described in Peten. et al. J. Exp. Med., 176: 1571-1576 (1992), unilateral nephrectomy specimens with renal carcinoma were obtained from human patients. The patients had no history of diabetes, hypertension or other systemic diseases associated with glomerular disease. Samples of cortical tissue distant from obvious tumor were placed in Carnoy's fixative, embedded in methacrylate or paraffin, and sections were stained with periodic acid-Schiff(PAS). The presence of glomerulosclerosis, defined as an expansion of the mesangial matrix, was independently evaluated by histological examination of PAS-stained material (FIG. 2, top panel) and by immunofluorescence microscopy of frozen sections after exposure to an antibody to type IV collagen (PHM-12, Silenus, Westbury, N.Y.) (FIG. 2, middle panel).

The competitive PCR assay was conducted as described in Example 1 to quantify the amount of α₂IV (scarring-type) extracellular matrix collagen. The relative concentrations of that collagen type in glomeruli previously found to be normal or sclerotic were determined, as shown in the lower panel of FIG. 2.

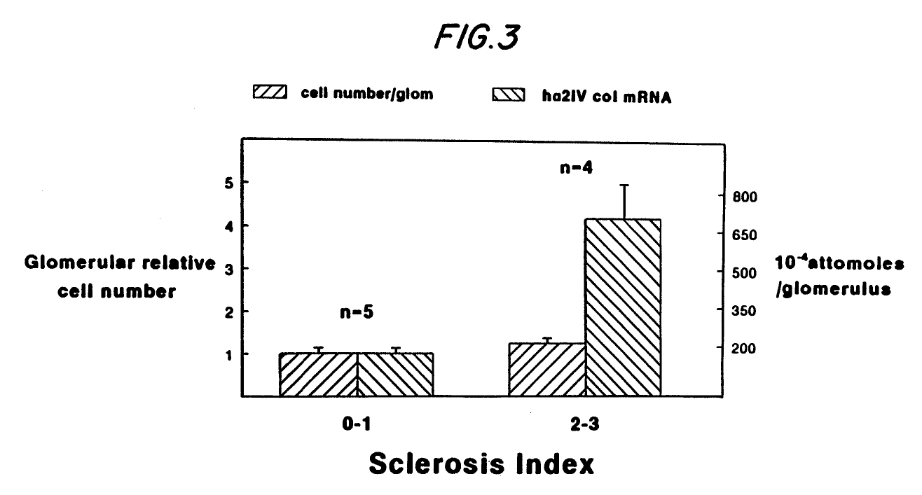

The relative cell numbers in glomeruli of five patients without glomerular sclerosis (normal) were compared to five patients with sclerosis. As reflected in FIG. 3 the difference between the groups in glomerular relative cell number was not significant (p>0.8) whereas, for the α₂IV collagen cDNA levels, the difference was statistically significant (0.01<p<0.025).

### EXAMPLE 3

### RELATIVE COLLAGEN mRNA RATIOS IN GLOMERULI FROM NORMAL AND DISEASED KIDNEYS

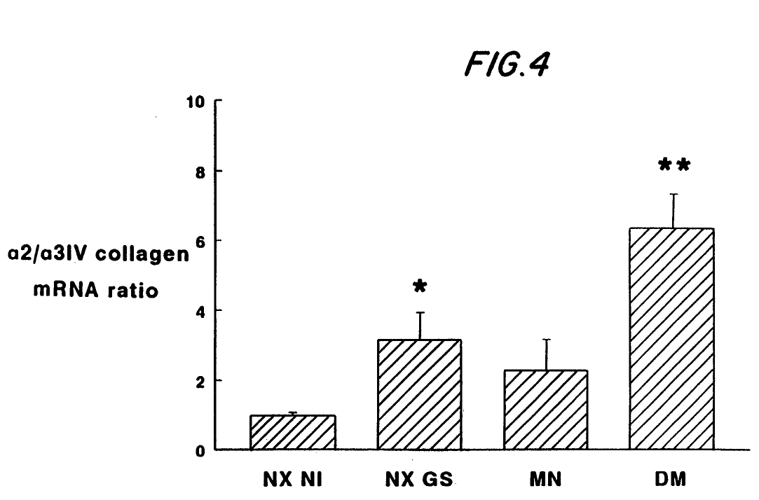

Utilizing the methodology described in Examples 1 and 2, the relative ratios of α2/α₃ IV collagen mRNA were quantified in glomeruli taken from diagnostic biopsies of human patients with membranous glomerulonephritis (MN) and diabetic nephropathy (DM) and from nephrectomies with glomerulosclerosis (NX GS) and without glomerulosclerosis (NX M1). As reflected in FIG. 4, the α₂/α₃IV collagen mRNA ratios were significantly higher in DM and in NS GS than in NX N1. (** P=0.0002, *P=0.02).

### EXAMPLE 4

### IN VITRO STUDIES WITH PPS

Study A

Experimental Design

Normal mesangial cells (8) were plated in basal medium plus 20% fetal bovine serum (Gibco, Grand Island, N.Y.) in 24-well plates (Nunc, PGC Scientific Corp., Gaithersburg, Md.) at a density of 2−2.5×10⁴ cells/well. At 24 hours the medium was discarded, cells were washed twice with PBS and incubated for 24-72 h in serum-free medium with 0.1% bovine serum albumin (RIA grade, Sicjma). The medium was replaced with fresh basal medium plus 20% fetal bovine serum with or without 5-100 μg/ml of PPS or compared to standard heparin (100 μg/ml). Cells of duplicate wells were trypsinized and counted in an Elzone® cell counter (Particle Data Inc., Elmhurst, Ill.) at days +3 and +5. In parallel wells, thymidine incorporation was determined by adding 1 μCi/well of [³H] thymidine ([methyl-³H] thymidine); 2.0 Ci/mM; DuPont NEN, Boston, Mass.). Counts were determined at day 1 or at day 3.

Results:

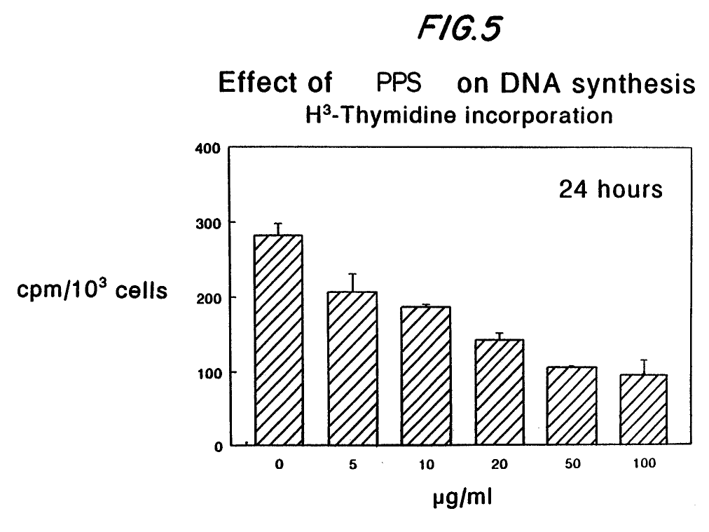

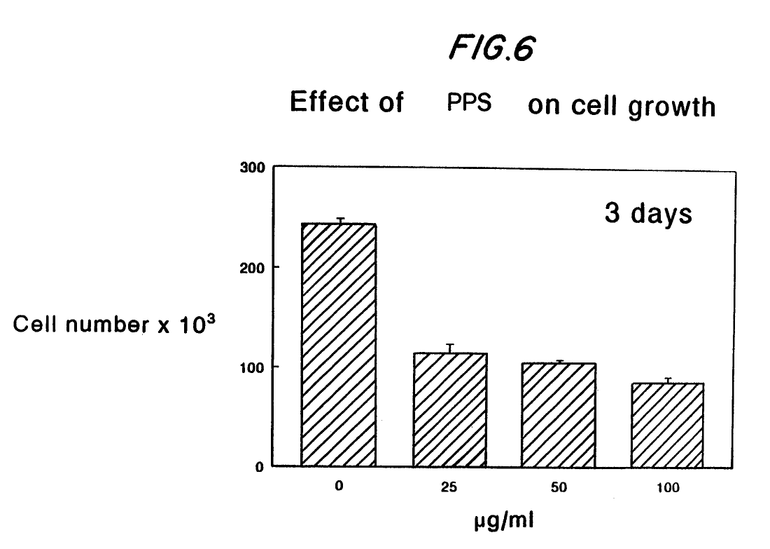

At day one (24 hours) the maximum dose-response plateaued at 50 μg/ml (FIG. 5) whereas at day three the maximum inhibitory response was noted at 25 μg/ml (FIG. 6).

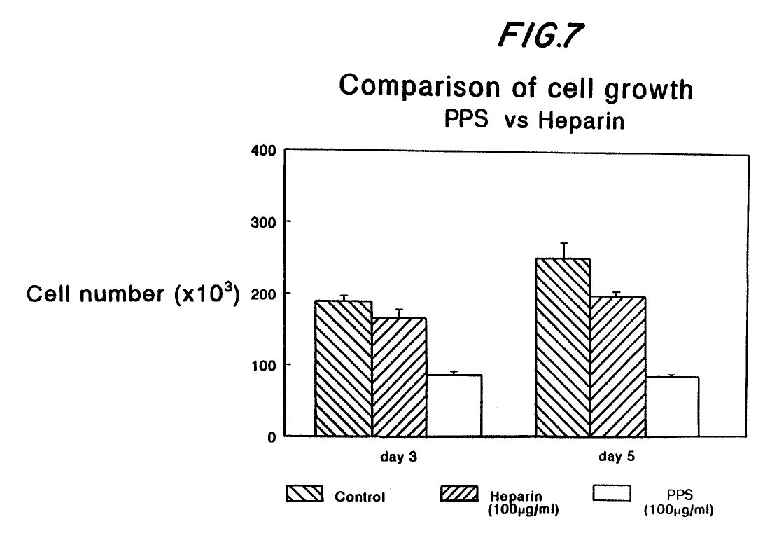

Comparison between no addition (control) and heparin (100 μg/ml) and PPS (100 μg/ml), reveals that on a molar basis PPS is roughly twice as potent as native heparin (FIG. 7). The responses are quite reproducible (the error bars are very tight).

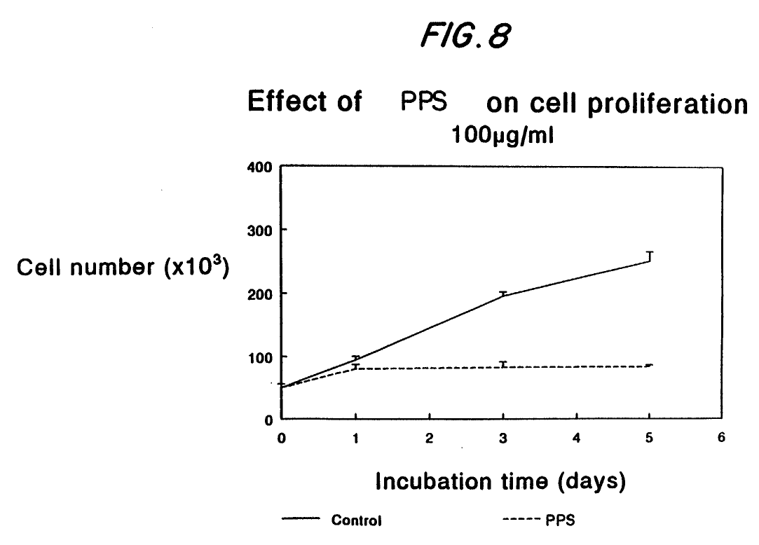

A summary graph (FIG. 8) compares the effect of PPS added to serum to control cells which were exposed only to serum.

Study B

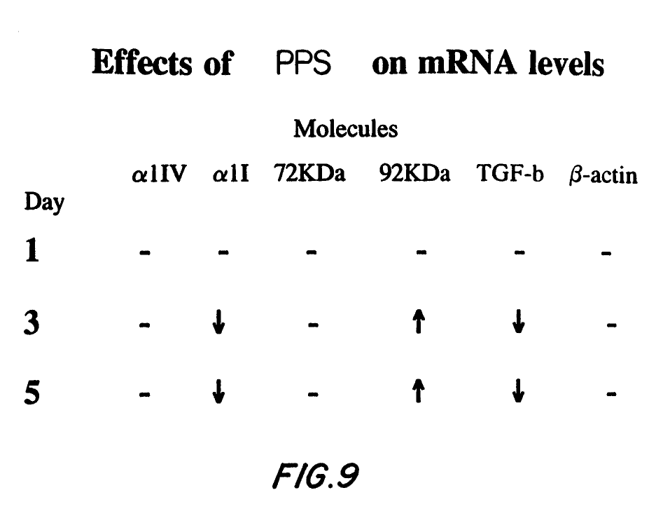

Normal mesangial cell layers were exposed to PPS (100 μg/ml) for varying periods, and reverse-transcribed, mRNA levels were measured for selected molecules at day 1 and compared with the levels at days 3 and 5 (see FIG. 9), There were no changes in type IV collagen mRNA, type I collagen MRNA was substantially decreased, TGF-β mRNA was reduced by 50%, and the 92 kDa enzyme activity was increased by more than 50%. The control was β-actin, which was unchanged, consistent with the absence of proliferation in the treated cells.

### EXAMPLE 5

### STUDIES WITH GH TRANSGENIC MICE

Experimental Design

Twelve 6-week old GH transgenic mice were identified by PCR analysis of detergent-extracted material from tail biopsies using specific primers for the bovine growth hormone cDNA that did not cross-react with the mouse GH sequence. Six GH mice were treated for 10-12 weeks with oral PPS sodium (Elmiron®, Baker Norton Pharmaceuticals, Inc.) in their drinking water and six age-matched GH mice received tap water for the same duration. The amount of PPS sodium in the drinking water was about 100 mg/kg of animal body weight.

Isolation of Glomeruli. and in situ Reverse Transcription

Glomeruli were isolated by microdissection in the presence of RNase inhibitors. The left kidney was perfused with saline followed by a collagenase solution containing soluble RNase inhibitors. The lower pole was removed prior to collagenase perfusion and snap frozen on dry ice for zymography. After collagenase digestion, 40-60 glomeruli were isolated at 4° C. in presence of vanadyl ribonucleoside complex, for reverse transcription (RT). In situ RT was performed as above except that the glomeruli were freeze-thawed once in acetone dry ice and sonicated at 2° C. for 5 minutes in the presence of 2% Triton and 4 units/μl of human placental RNase inhibitor (Boehringer Mannheim, Indianapolis, Ind.), prior to the addition of the RT components. A Micro Ultrasonic Cell Disrupter (Kontes, Vineland, N.J.) was used to refrigerate the samples during sonication.

Standard and Competitive PCR Assays

Primers for mouse α₁IV and α₁I collagen, α smooth muscle cell actin, β-actin, laminin B1, tenascin, 92 kDa metalloproteinase and 72 kDa metalloproteinase mRNAs, and for bovine growth hormone genomic DNA, were synthesized on a PCR-Mate (Applied Biosystems, Foster City, Calif.). The identity of each amplified product was verified by size and by restriction enzyme analysis. Primer specificity for mRNA was determined by omitting the reverse transcriptase enzyme. PCR was performed Using the GeneAmp DNA Amplification kit (Perkin Elmer Cetus, Norwalk, Conn.). cDNA derived from a pool of 40-60 glomeruli/mouse was initially assayed by standard PCR, using the log-linear part of PCR amplification. This permitted a rapid, non-quantitative assessment of mRNA levels. Thereafter, competitive PCR assays were utilized to measure α₁IV collagen (and the ratio of α₁IV collagen to GAPDH enzyme was calculated to normalize the data between animals), PDGF-B, α smooth muscle cell actin, β-actin, and laminin B1 cDNAs by constructing a cDNA mutant for each molecule, with a small internal deletion or a new restriction enzyme site. Analysis of PCR products was performed using a PDI densitometer loaded with the Quantity One® image analysis software. Competitive PCR assays were performed in duplicate or triplicate.

Results

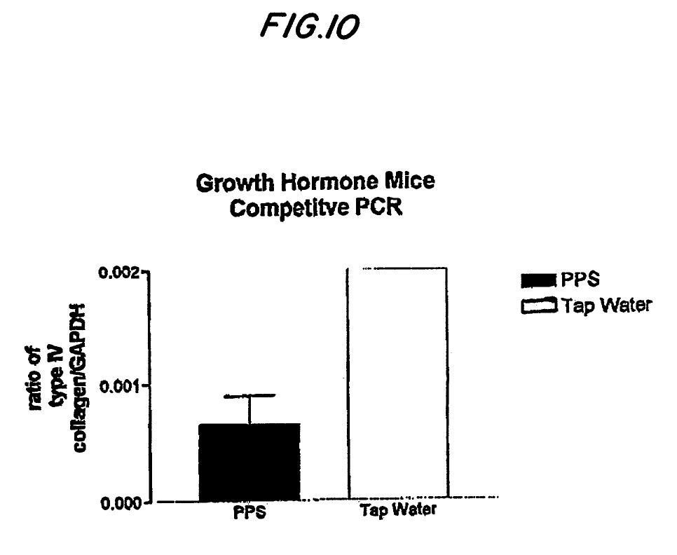

As shown in FIG. 10, the mean type IV collagen/GAPDH ratio was less than half in the group of mice treated with oral PPS sodium than in the mice of the untreated (control) group. This differential indicates that considerably less scarring-type collagen was present in the glomeruli of the treated animals in comparison with the untreated animals, a fact which was confirmed by histological examination and immunofluorescent microscopy.

### EXAMPLE 6

### STUDIES WITH WATANABE RABBITS

Watanabe rabbits¹ serve as an animal model of natural endogenous hypercholesterolemia. This trait is completely expressed in the homozygous state, is partly expressed in the heterozygous state and is due to a single-gene defect. Homozygous Watanabe rabbits have serum cholesterol concentrations 8 to 14 times greater than normal Japanese white rabbits. ¹ This strain of rabbits is technically known as the Watanabe heritable hyperlipidemic rabbit (WHHL).

Watanabe rabbits have a very high incidence of atherosclerotic plaques, particularly in the aorta. The rapidity of development and severity of the atherosclerosis can be increased by feeding the rabbits a diet high in cholestrol.

The following two studies were conducted to ascertain the anti-atherosclerotic activity of PPS in Watanabe rabbits:

Study A: Subcutaneous Evaluation of PPS

Twelve Watanabe rabbits were divided into two groups of six each (Group A and Group B) and fed a high cholesterol diet (0.5% cholesterol). The animals of Group A were treated daily with normal saline subcutaneously, while the animals of Group B were treated daily with 10 mg/kg of PPS sodium (Elmiron®) subcutaneously.

Four of the PPS-treated animals (Group B) died prior to completion of the study, one on day 22 and three between day 80 and day 86. On day 89, the animals of Group A and the two remaining animals of Group B were euthanized and necropsied and their tissues evaluated, particularly sections from different major branches of the aorta.

Results

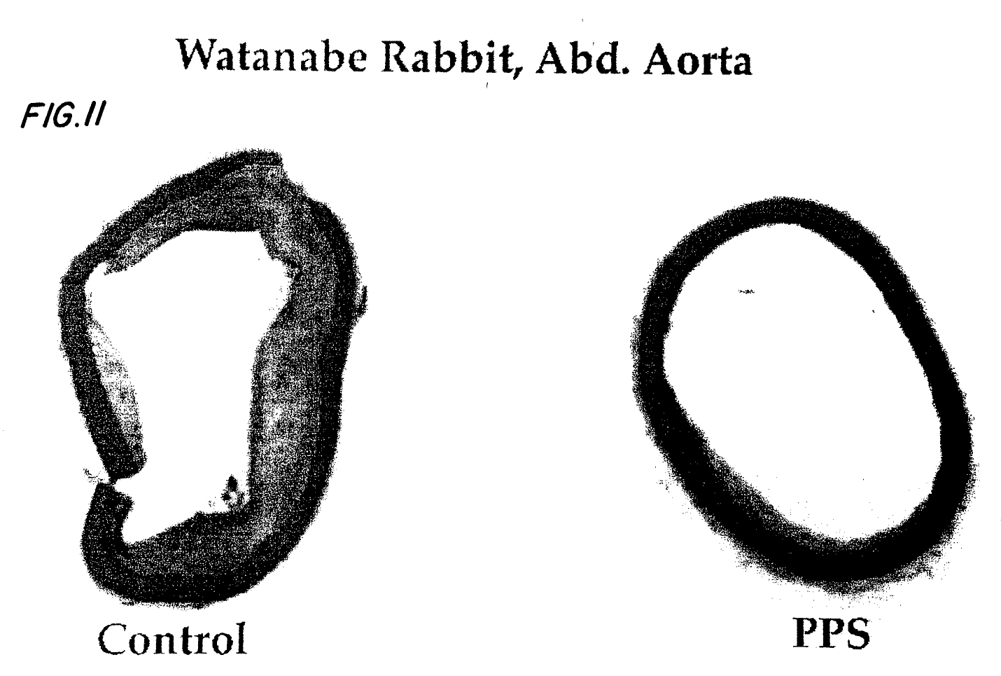

As shown in Table I below, the animals of the treatment Group B were found to have much smaller plaque deposits and a much higher ratio of smooth muscle layer to plaque (as much as 6.8 times higher) in comparison with the control rabbits of Group A in all of the aortal cross-sections examined. These findings are visually illustrated in photographs shown in FIG. 11. The cross-section of the abdominal aorta from a control group animal shows highly developed aterosclerotic plaque of substantial cross-sectional area. The cross-section from the abdominal aorta of an animal treated with PPS sodium shows almost no sign of plaque although the treatment group animals were fed the same high cholesterol diet as the control group.

TABLE I
WATANABE RABBITS
Morphometry of Aortic Lesions

| | | | | | | Fold |
|---|---|---|---|---|---|---|
| | | | Smooth | | Smooth | decrease in |
| | | Control | Muscle/ | PPS | Muscle/ | Plaque Size |
| | | (cm²) | Plaque | (cm²) | Plaque | (×) |
| Ascending | Smooth | 0.328 | 0.61 | 0.243 | 4.12 | 6.8× |
| Aorta | Muscle | | | | | |
| | Layer | | | | | |
| | Plaque | 0.54 | | 0.59 | | |
| Aortic Arch | Smooth | | | | | |
| | Muscle | | | | | |
| | Layer | | | | | |
| | Plaque | | | | | |
| Thoracic | Smooth | 0.244 | 0.47 | 0.334 | 1.184 | 2.5× |
| Aorta | Muscle | | | | | |
| | Layer | | | | | |
| | Plaque | | | | | |
| Abdominal | Smooth | 0.265 | 0.74 | 0.303 | 7.58 | 10.2× |
| Aorta | Muscle | | | | | |
| | Layer | | | | | |
| | Plaque | 0.358 | | 0.04 | | |

Study B: Oral Evaluation of PPS

Twenty Watanabe rabbits were divided into four groups of five each, Groups A through D. All of the rabbits were fed the same high cholesterol diet (0.5% cholesterol). The animals of Groups A and B were given tap water to drink, while the animals of Groups C and D were given tap water containing 0.5 mg/mL of PPS sodium (Elmiron®). Based on observations of pre-study water consumption by the animals, the total daily dose of PPS sodium consumed by each animal in the treatment groups was about 30 mg/kg.

Two of the treated rabbits were removed from the study on days 4 and 11, respectively, due to abscesses apparently unrelated to the PPS.

The animals of Groups A and C were euthanized and necropsied on day 50 of the study and their aortae examined. Significant differences were observed visually between the intima of Group A (control) animals and those of Group C (treated) animals, with the latter exhibiting less atherosclerotic plaque development.

The rabbits of Groups B and D were euthanized and necropsied on day 64 of the study. The aortae of these groups were examined histologically and the respective cross-sectional areas of the intimal and medial layers in various aortal branches were measured.

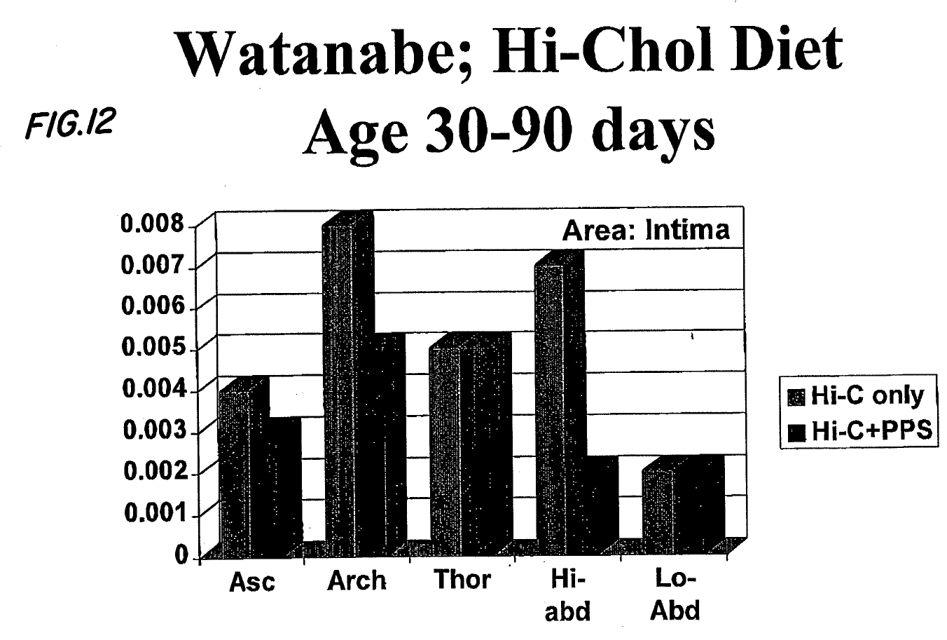

FIG. 12 is a bar graph reflecting the mean intimal areas measured in cross-sections taken from various branches of the aortae of the rabbits of the control group (Group B) and the treatment group (Group D), respectively. FIG. 12 illustrates that in each aortal branch examined the intimal area was substantially less in the treated animals as compared with the untreated ones, indicating that there were substantially less atherosclerotic lesions and plaque deposits in the vasculature of the treatment group.

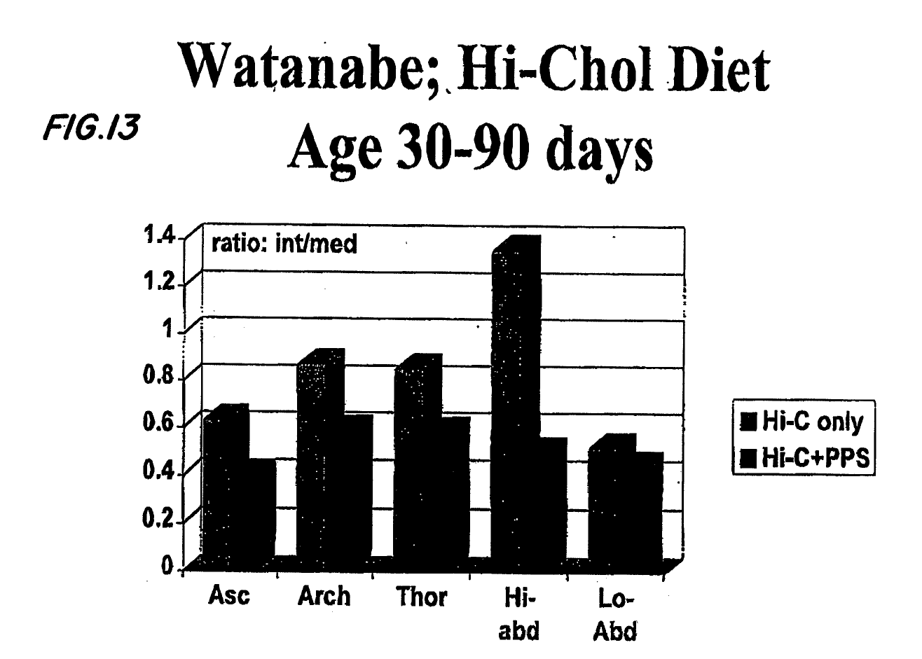

FIG. 13 shows the mean values for the ratio of intima to medial areas in the same aortal cross-sections taken from Group B and D rabbits as described with respect to FIG. 12. This ratio, which is a reflection of the relative amount of scar tissue and plaques deposited on the vessel walls (which deposits increase the cross-sectional area of the intima), was lower in every aortal branch of the treated rabbits (Group D) in comparison with the untreated animals (Group B).

The foregoing data, generated by scientifically validated experimental procedures, demonstrate the effectiveness of PPS in decreasing the synthesis of excess extracellular matrix collagen and certain cellular growth factors while increasing the activity of collagen degradation enzymes. These effects indicate that PPS should be highly effective in the clinical management and reversal of CPVSD, particularly arteriosclerosis and atherosclerosis.

It has thus been shown that there are provided methods and compositions which achieve the various objects of the invention and which are well adapted to meet the conditions of practical use.

As various possible embodiments might be made of the above invention, and as various changes might be made in the embodiments set forth above, it is to be understood that all matters herein described are to be interpreted as illustrative and not in a limiting sense.

What is claimed as new and desired to be protected by Letters Patent is set forth in the following claims.

We claim:

1. A method of treating a mammalian patient suffering from a chronic progressive vascular scarring disease (CPVSD) in an affected vasculature which causes narrowing of the lumen thereof and reduction of distensibility, to halt the progress of the disease and cause the resolution or diminution of already-formed scarring lesions, said method consisting of the administration to the patient of a pharmaceutical composition containing an effective vascular scarring disease treatment amount of pentosan polysulfate (PPS) or a pharmaceutically acceptable salt thereof.

2. A method according to claim 1 wherein the affected vasculature is an artery.

3. A method according to claim 2 wherein said artery is the aorta or a major branch thereof.

4. A method according to claim 2 wherein said disease is a form of arteriosclerosis characterized by scarring and wherein the arteriosclerotic scarring process is reversed by said method.

5. A method according to claim 4 wherein said form of arteriosclerosis is atherosclerosis and said scarring involves arterial walls affected by atherosclerotic plaques.

6. A method according to claim 1 wherein a sufficient amount of said pharmaceutical composition is administered to the patient to provide a total daily dose of about 5 to about 30 mg/kg of patient body weight or about 350 to about 2,000 mg of PPS or a pharmaceutically acceptable salt thereof.

7. A method according to claim 6 wherein said daily dosage is about 500 to about 1,500 mg.

8. A method according to claim 6 wherein said daily dosage is administered in one to four equally divided doses.

9. A method according to claim 1 wherein said pharmaceutical composition is an orally administered dosage form.

10. A method according to claim 9 wherein said dosage form is selected from the group consisting of conventional or sustained release tablets, coated tablets, capsules, caplets, lozenges, liquids and elixirs.

11. A method according to claim 9 wherein said dosage form includes at least one pharmaceutically acceptable inert ingredient.

12. A method according to claim 11 wherein said inert ingredient is a filler, binder, solvent, excipient or carrier.

13. A method according to claim 9 wherein said dosage form contains about 50 to about 300 mg per unit of PPS or a pharmaceutically acceptable salt thereof.

14. A method according to claim 1 wherein said pharmaceutically acceptable salt is the sodium salt.

15. A method according to claim 14 wherein said composition is in the form of a gelatin capsule containing PPS sodium, microcrystalline cellulose and magnesium stearate.

16. A method according to claim 1 wherein said patient is a human patient.
